(19) United States
(12) Patent Application Publication
(10) Pub. No.: US 2021/0015392 A1
(43) Pub. Date: Jan. 21, 2021

(54) DEEP INTRACRANIAL ELECTRODE, ELECTROENCEPHALOGRAPH AND MANUFACTURING METHOD THEREOF

(71) Applicant: NEUROECHOS MEDICAL (SHENZHEN) CO., LTD, Shenzhen (CN)

(72) Inventors: Xijun GUAN, Shenzhen (CN); Xiaolong MO, Shenzhen (CN)

(21) Appl. No.: 16/549,650

(22) Filed: Aug. 23, 2019

Related U.S. Application Data
(63) Continuation of application No. PCT/CN2019/096389, filed on Jul. 17, 2019.

(51) Int. Cl. A61B 5/0478; A61N 1/05; A61N 1/36; A61B 18/14; A61B 5/00

(52) U.S. Cl. CPC A61B 5/0478; A61N 1/0531; A61N 1/36064; A61B 2017/00867; A61B 18/1492; A61B 5/4094; A61N 1/0534

(57) ABSTRACT

A method for manufacturing a deep intracranial electrode, a bending-resistant deep intracranial electrode and an electroencephalograph is disclosed. The method comprises the following steps: manufacturing a support rod of the deep intracranial electrode with a shape memory alloy material, the shape memory alloy having a preset phase-transformation temperature; subjecting the support rod in a straight state to an annealing process such that the support rod memorizes a straight shape.

### CROSS-REFERENCE TO RELATED APPLICATION

This application is a continuation of PCT application No. PCT/CN2019/096389, filed on Jul. 17, 2019. The patent application is hereby incorporated by reference in its entirety.

### TECHNICAL FIELD

The present disclosure relates to the field of medical apparatuses and equipment, more particularly, to a method for manufacturing a bending-resistant deep intracranial electrode, a bending-resistant deep intracranial electrode and an electroencephalograph.

### BACKGROUND

SEEG (Stereoelectroencephalography) is short for three-dimensional electroencephalography. This technique leads the localization method from 2D to 3D. Based on clinical symptoms-cortical discharge-neural anatomy, the imaging technique covers the brain in an all-round three-dimensional mode using a stereotactic technique, so as to locate the lesion accurately and improve the therapeutic effect.

Patients with intractable epilepsy require a preoperative assessment. Existing non-invasive diagnosis and treatment cannot determine the location of an epileptic foci. In order to better monitor neurological activities of the brain directly with high resolution, it is necessary to put electrodes into the skull for intracranial electroencephalography monitoring. Intracranial electroencephalography may eliminate scalp and skull interference by placing electrodes in the sulcus or deep brain. SEEG may directly place the electrodes to targeted intracranial areas, such as deep frontal lobe, inner side of brain, cingulate gyms, medial temporal lobe and other areas that cannot be reached by conventional cortical electrodes. Minimally invasive method is adopted to set up an electrode path before operation for avoiding intracranial arteries and veins and protecting brain functions maximally.

Deep intracranial electrodes are valuable adjuvant treatments to refractory epilepsy surgery and are used to record epileptic foci source discharge from a deep brain tissue of epileptic patients. It helps identify areas and epileptic foci of interparoxysmal dysfunction, and may be used to determine the intensity and range of abnormal discharge in the deep cerebral cortex of the suspected cortex, as shown in FIG. 6.

Since the deep intracranial electrodes are in direct contact with the cerebral cortex, electrical signals collected by the electrodes may directly reflect real physiological activities in surrounding brain areas. Therefore, the stereotactic technique may monitor neural electrical activities of the cortex with high spatial and temporal resolutions, which makes the stereotactic technique play an irreplaceable role in both clinical epileptic foci localization and basic research on brain science, and possess many advantages over scalp electroencephalography.

However, the existing deep intracranial electrodes still have the following issues:

Existing electroencephalography products usually use medical apparatuses and equipment with magnetic metal materials, such as stainless steel, to manufacture conductors and electrode contacts of the deep intracranial electrodes. The medical apparatuses and equipment using magnetic materials are not compatible with high field (3.0 T) magnetic resonance imaging equipment. Magnetic metal materials may interfere with the magnetic field environment of the magnetic resonance imaging equipment (MRI), resulting in image artifacts and affecting diseases diagnosis. In addition, internal structures of a deep intracranial electrode include a slender conductor. In magnetic resonance imaging, the conductor will absorb radio-frequency magnetic field energy generated by the equipment, and generate energy deposition at electrode contacts, resulting in heating of the electrode contacts, which may damage brain tissues and endanger the life and health of the patients.

Because the deep intracranial electrodes themselves are very slim and small, structural strength thereof is low and tensile strength thereof is not high. In the process of long-term continuous electroencephalography detection, the electrodes are prone to be pulled out of the brain or pulled off unexpectedly.

In addition, to-be-implanted parts of the deep intracranial electrode are prone to accidental bending due to improper operation, which cannot be recovered after bending and may only be scrapped. Alternatively, to ensure the accuracy of electrode implantation, a rod-shaped slender support with certain stiffness is arranged inside the front end of the electrode to ensure that the end of the electrode remains straight. However, the support is usually made of tungsten or aluminum alloy and other metal materials. During the use of the electrode, improper operations may easily bend the front end of the electrode, and it cannot be recovered to the original straight state after bending, scrapping the product.

Therefore, the existing deep intracranial monitoring technology requires to be improved and developed.

### SUMMARY

In view of the above technical issues, a method for manufacturing a deep intracranial electrode, a bending-resistant deep intracranial electrode and an electroencephalograph, which provide special protection measures for the electrode and may recover the electrode to an original shape after being deformed by external force by virtue of good bending resistance thereof when collecting patients' deep electrophysiological signals, are provided.

Firstly, a technical solution provided by an embodiment of the present disclosure is providing a method for manufacturing a bending-resistant deep intracranial electrode, which comprises:

Manufacturing a support rod of the deep intracranial electrode with a shape memory alloy material, the shape memory alloy having a preset phase-transformation temperature;

Subjecting the support rod in a straight state to an annealing process such that the support rod memorizes a straight shape.

In one embodiment, the shape memory alloy material is a nickel-titanium shape memory alloy, the preset phase-transformation temperature being a first phase-transformation temperature higher than a storage and ambient temperature of the deep intracranial electrode;

When the deep intracranial electrode is deformed, heating up the deep intracranial electrode to be over the first phase-transformation temperature to recover the support rod of the deep intracranial electrode to an original straight shape.

In another embodiment, the shape memory alloy material is a nickel-titanium shape memory alloy, the preset phase-transformation temperature being a second phase-transformation temperature lower than a storage and ambient temperature of the deep intracranial electrode;

when the deep intracranial electrode is deformed, standing the deep intracranial electrode for a preset time to recover the support rod of the deep intracranial electrode to an original straight shape.

Secondly, a technical solution provided by an embodiment of the present disclosure is providing a bending-resistant deep intracranial electrode, which comprises an intracranial electrode support device, a plurality of electrode contacts and a flexible catheter, the intracranial electrode support device comprising an insulated support rod and a flexible sleeve, the plurality of electrode contacts fixed outside the flexible sleeve, wherein the support rod is installed inside the flexible sleeve, and a gap receiving conducting wires of the plurality of electrode contacts is defined between the support rod and the flexible sleeve; the support rod is made of a shape memory alloy material which is subjected to an annealing process and with a preset phase-transformation temperature such that the support rod recovers to an original shape after being deformed by an external force.

In order to reduce the harm of resonance heating to the patients, the medicinal shape memory alloy material is a non-magnetic shape memory alloy material.

As a first embodiment, the shape memory alloy material is a nickel-titanium shape memory alloy, the preset phase-transformation temperature being a first phase-transformation temperature higher than a storage and ambient temperature of the deep intracranial electrode.

As in another embodiment, the shape memory alloy material is a nickel-titanium shape memory alloy, the preset phase-transformation temperature being a second phase-transformation temperature lower than a storage and ambient temperature of the deep intracranial electrode.

Where the bending-resistant deep intracranial electrode further comprises a connector connecting the flexible catheter and a shield sleeve, the flexible catheter being folded and received in the shield sleeve; by pulling out a preset length of the flexible catheter from the shield sleeve, a conductor length is varied and a resonant heating of the bending-resistant deep intracranial electrode is reduced.

A plurality of electrode conductors of the plurality of electrode contacts are received in the flexible catheter, each electrode contact being electrically connected to corresponding connection terminal of the connector via an electrode conductor.

The intracranial electrode supporting device is connected to the flexible catheter via a guiding fixing assembly.

In head localization and fixation, the guiding fixing assembly comprises a guiding fixing screw and a guiding fixing nut which are for clasping and connecting the support rod, the flexible sleeve and the flexible catheter.

In order to lessen force applied to the electrode conductors, a tensile fiber is disposed between each electrode contact on the electrode support device and a corresponding connecting terminal of the connector.

In order to further lessen force applied to the electrode conductors, a length of the flexible catheter is less than that of an electrode body within the flexible catheter.

Thirdly, a technical solution provided by an embodiment of the present disclosure is providing an electroencephalograph, connected to a plurality of deep intracranial electrodes, each deep intracranial electrode comprising an intracranial electrode support device, a plurality of electrode contacts and a flexible catheter, the intracranial electrode support device comprising an insulated support rod and a flexible sleeve, the plurality of electrode contacts fixed outside the flexible sleeve, wherein the support rod is installed inside the flexible sleeve, and a gap receiving conducting wires of the plurality of electrode contacts is defined between the support rod and the flexible sleeve; the support rod is made of a shape memory alloy material which is subjected to an annealing process and with a preset phase-transformation temperature such that the support rod recovers to an original shape after being deformed by an external force.

In order to avoid resonant heating, the medicinal shape memory alloy material is a non-magnetic shape memory alloy material.

As a first embodiment, the shape memory alloy material is a nickel-titanium shape memory alloy, the preset phase-transformation temperature being a first phase-transformation temperature higher than a storage and ambient temperature of the deep intracranial electrodes.

As in another embodiment, the shape memory alloy material is a nickel-titanium shape memory alloy, the preset phase-transformation temperature being a second phase-transformation temperature lower than a storage and ambient temperature of the deep intracranial electrodes.

In order to improve compatibility with the magnetic resonance imaging equipment, each of the bending-resistant deep intracranial electrodes further comprises a connector connecting the flexible catheter and a shield sleeve, the flexible catheter being folded and received in the shield sleeve; by pulling out a preset length of the flexible catheter from the shield sleeve, a conductor length is varied and a resonant heating of the bending-resistant deep intracranial electrode is reduced.

Beneficial effects of embodiments of the present disclosure include: in the method for manufacturing the deep intracranial electrode, the bending-resistant deep intracranial electrode and the electroencephalograph provided by the embodiments, the support rod in an implanted end of the electrode is made of a shape memory alloy material. While collecting the deep electrophysiological signal from the patients, the support rod provides the bending-resistant deep intracranial electrode with special protective measures, and makes the implanted end of the electrode recover to its original shape after being deformed by external force, improving bending-resistant capability of the implanted end of the electrode and prolonging service life of the medical apparatus and equipment.

In the method for manufacturing the deep intracranial electrode, the bending-resistant deep intracranial electrode and the electroencephalograph provided by the embodiments, the support rod of the implanted end of the electrode is made of a non-magnetic shape memory alloy material, which is compatible with high field magnetic resonance imaging operations, such that the bending-resistant deep intracranial electrode is implanted, at the same time, high field magnetic resonance imaging is performed simultaneously. For example, 3.0 TMRI compatibility is realized, and artifacts in magnetic resonance imaging caused by the electrode is also eliminated.

### BRIEF DESCRIPTION OF THE DRAWINGS

One or more embodiments will be described for exemplary purpose in accompany with corresponding drawings, which descriptions do not constitute limitation to embodiments of the present disclosure. Like reference numbers labeled in the drawings indicate similar components. Unless otherwise indicated, the drawings do not constitute limitation to the present disclosure.

FIG. 1 is a structural schematic view of a bending-resistant deep intracranial electrode according to an embodiment of the present application.

FIG. 2 is a structural view of an implanted head end and a flexible catheter of the bending-resistant deep intracranial electrode according to an embodiment of the present application.

FIG. 3 is a perspective breakdown view of a support device of the bending-resistant deep intracranial electrode according to an embodiment of the present application.

FIG. 4 is a structural schematic view of a head end of the bending-resistant deep intracranial electrode according to an embodiment of the present application.

FIG. 5 is a structural schematic view of the support device of the bending-resistant deep intracranial electrode according to an embodiment of the present application.

FIG. 6 is an intracranial imaging schematic view of an electroencephalograph according to an embodiment of the present application.

FIG. 7 is a curve graph of a length of a short conductor versus resonance heating of the bending-resistant deep intracranial electrode according to an embodiment of the present application.

FIG. 8 is a schematic view of clinical application of a conductor of the bending-resistant deep intracranial electrode received into a shield sleeve.

FIG. 9 is a schematic curve graph of a length of a long conductor versus resonance heating of the bending-resistant deep intracranial electrode according to an embodiment of the present application.

FIG. 10 is a schematic view of clinical application of a conductor of the bending-resistant deep intracranial electrode pulled completely out from the shield sleeve.

FIG. 11 is a flow chart of a method for manufacturing the bending-resistant deep intracranial electrode.

### DETAILED DESCRIPTION OF EMBODIMENTS

Technical solutions in the embodiments of the present disclosure will be described clearly and completely with reference to the drawings. Obviously, the embodiments described below are only some, but no exclusive of the embodiments of the present disclosure. Based on the embodiments described in this present disclosure, all other embodiments obtained by those ordinarily skilled in the field without paying creative works should fall within the scope of the present application.

It should be noted that any directional indication (such as top, bottom, left, right, front, back . . . ) involved in the embodiment of the present application is only used to explain relative location relations and motion among each component under a specific position (as shown in the drawings). If the specific position varies, the directional indication varies accordingly.

Furthermore, the terms “first”, “second” and the like involved in the embodiment of the present application are merely for illustrative purpose, but not intended to indicate or imply relative importance, or imply the number of associated features. Therefore, the features limited by “first” and “second” may explicitly or implicitly include at least one of these features. In addition, the technical solutions among each embodiment may be combined with each other, however, the combination must be capable of being realized by those ordinarily skilled in the field. When the combination of technical solutions is contradictory or cannot be realized, the combination of such technical solutions shall not be deemed as existing and shall not fall within the scope of the present application.

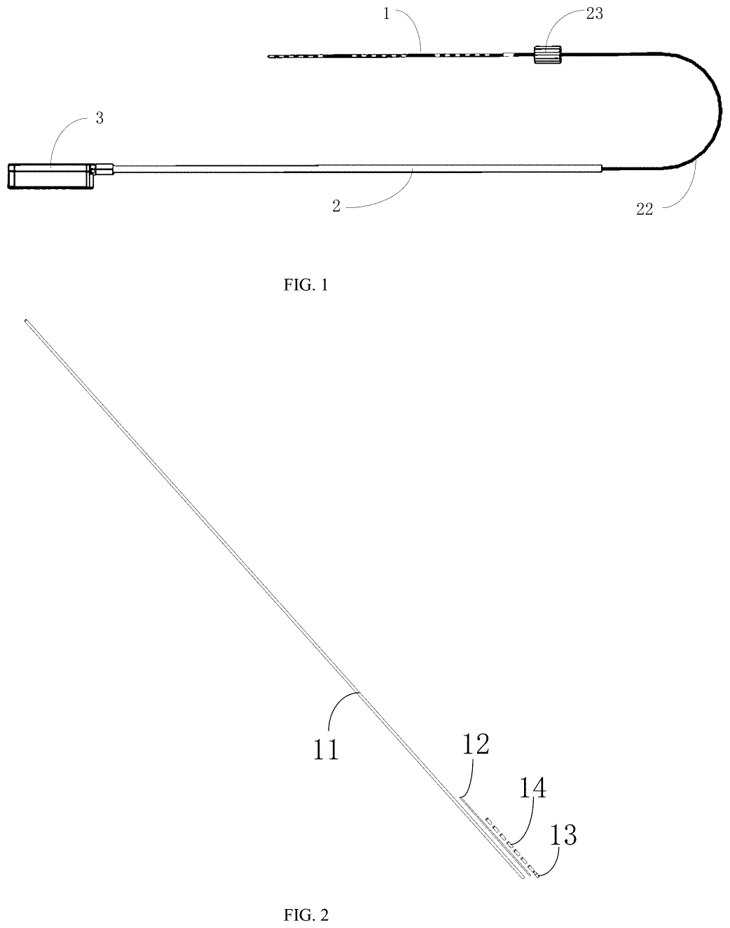

Referring to FIG. 1, an embodiment concerns a method for manufacturing a bending-resistant deep intracranial electrode, a bending-resistant deep intracranial electrode and an electroencephalograph.

The bending-resistant deep intracranial electrode is inserted into the patient's targeted intracranial area through minimally invasive surgery like craniotomy or drilling. For example, the scalp and skull are surgically perforated with a 2 mm micropore, and the deep electrode is placed in the targeted area deep in the deep brain. Based on three-dimensional brain network concept integrating anatomy, electricity and clinic, the epileptic foci are explored and located with aid of the stereoelectroencephalography, as shown in FIG. 6. The stereoelectroencephalography involves three-dimensional reconstruction of cerebral cortex, three-dimensional reconstruction of cerebrovascular, skull MRI, CT angiography, PET-CT and other imaging fusion technologies. The stereoelectroencephalography further involves associated surgical hardware equipment and electrode implantation plan systems. The stereoelectroencephalography is a safe, reliable and minimally invasive intracranial electrode implantation system which has been verified in international clinical application.

In the method for manufacturing the deep intracranial electrode, the bending-resistant deep intracranial electrode and the electroencephalograph provided by the embodiment, the support rod 12 in an implanted end of the electrode is made of a shape memory alloy material. While collecting the deep electrophysiological signals from the patients, the support rod 12 provides the bending-resistant deep intracranial electrode with special protective measures, and makes the implanted end of the electrode recover to its original shape after being deformed by external force, improving bending-resistant capability of the implanted end of the electrode and prolonging service life of the medical apparatus and equipment.

The shape memory alloys (SMA) in the embodiment are materials made of two or more metal elements, and have shape memory effect (SME) processed by thermo-elastic and martensite phase-transformation and contravariants thereof.

The shape memory effect of the shape memory alloys results from the thermo-elastic martensite phase-transformation. Once the martensite is formed, it will continue to grow as the temperature drops, and decrease as the temperature rises, disappearing in a reversed process. The difference between the two free energies is the driving force for the phase-transformation. Another property of shape memory alloy is super elasticity. The shape memory alloy material possesses much better deformation recovery capacity than other metals under external force, that is, large strain generated in a loading process will recover along with an unloading process.

This embodiment adopts nickel-titanium shape memory alloys applied in medical field. In addition to taking advantage of shape memory effect or super elasticity thereof, the alloys also meet chemistry and biology requirements, which refers to good biocompatibility. The nickel-titanium shape memory alloys may form a stable passivation film with organisms.

### The First Embodiment

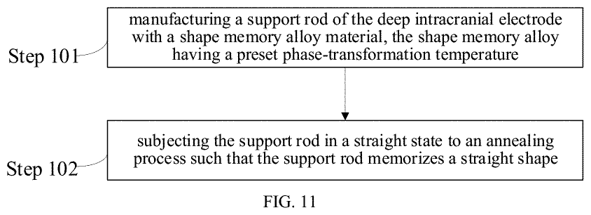

Referring to FIG. 11, a method for manufacturing a bending-resistant deep intracranial electrode provided by the embodiment comprises:

At block 101, a support rod of the deep intracranial electrode is manufactured with a shape memory alloy material. The shape memory alloy has a preset phase-transformation temperature.

At block 102, the support rod in a straight state is subjected to an annealing process such that the support rod memorizes a straight shape.

As a first embodiment, the shape memory alloy material is a nickel-titanium shape memory alloy (NiTi). The preset phase-transformation temperature is a first phase-transformation temperature higher than a storage and ambient temperature of the deep intracranial electrode.

The phase-transformation temperature Af of the shape memory alloy is associated with composition ratios of each element. By accurately adjusting the ratios of each element in the NiTi alloy, the phase-transformation temperature Af of the shape memory alloy can be higher than the storage and ambient temperature of the electrode. The first phase-transformation temperature is preferably 50° C. The shape memory alloy material is manufactured to be a slender support rod, which will memorize the current straight shape after being subjected to the annealing process.

When the deep intracranial electrode is deformed, the deep intracranial electrode is heat up to be over the first phase-transformation temperature to recover the support rod of the deep intracranial electrode to an original straight shape.

In the process of us, if the deep intracranial electrode is bent, only by blowing with hot wind or immersing in hot water may the front end of the electrode be heated up to over the first phase-transformation temperature Af of the shape memory alloy, and the support rod may recover to the original straight shape.

As a second embodiment, the shape memory alloy material is also a nickel-titanium shape memory alloy. The difference is that the preset phase-transformation temperature is a second phase-transformation temperature lower than a storage and ambient temperature of the deep intracranial electrode.

In a preferable embodiment, the second phase-transformation temperature may be 0° C. or −20° C. the slender support rod manufactured using the NiTi shape memory alloy materials with the second phase-transformation temperature and subjected to the annealing process may memorize current straight shape. In the second embodiment, the ambient temperature is over the second phase-transformation temperature Af of the NiTi shape memory alloy material, such that the support rod has super elasticity. The super elasticity indicates that even when the material is subjected to a plastic deformation beyond elastic limit thereof, the support rod may still recover to its original straight shape slowly.

When the deep intracranial electrode is deformed, the deep intracranial electrode is stood for a preset time for the support rod of the deep intracranial electrode to recover to the original straight shape. In the process of use, if the front end of the electrode is bent accidentally, only by standing the deep intracranial electrode for a preset time may the support rod recover to the original straight shape.

### The Second Embodiment

An electroencephalograph in the present embodiment is configured to monitor bioelectrical amplification of brain electrophysiological signals and an imaging equipment.

As shown in the drawings, the electroencephalograph is connected to a plurality of deep intracranial electrodes.

Each of the bending-resistant deep intracranial electrodes comprises an intracranial electrode support device 1, a plurality of electrode contacts 14, a flexible catheter 22, a connector 3 connected to the flexible catheter and a shield sleeve 2.

The intracranial electrode support device 1 comprises an insulated support rod 12, a flexible sleeve 11 and the plurality of electrode contacts 14.

The support rod 12 is installed inside the flexible sleeve 11. The plurality of electrode contacts 14 is fixed outside the flexible sleeve 11. A gap receiving conducting wires of the plurality of electrode contacts 14 is defined between the support rod 12 and the flexible sleeve 11. The support rod 12 is made of a shape memory alloy material which is subjected to an annealing process and with a preset phase-transformation temperature such that the support rod 12 recovers to an original shape after being deformed by an external force.

A plurality of electrode conducting wires of the plurality of electrode contacts 14 are received in the flexible catheter 22, each electrode contact being electrically connected to corresponding connection terminal of the connector 3 via the electrode conducting wires.

In order to avoid resonant heating, the medicinal shape memory alloy material is a non-magnetic shape memory alloy material.

As a first embodiment, the non-magnetic shape memory alloy material is a nickel-titanium shape memory alloy (TiNi). The preset phase-transformation temperature is a first phase-transformation temperature higher than a storage and ambient temperature of the deep intracranial electrodes.

As in another embodiment, the non-magnetic shape memory alloy material is a nickel-titanium shape memory alloy (TiNi). The preset phase-transformation temperature is a second phase-transformation temperature lower than the storage and ambient temperature of the deep intracranial electrodes.

In order to improve compatibility with the magnetic resonance imaging equipment, the length of bare conductor of the flexible catheter 22 of the bending-resistant deep intracranial electrode in the present embodiment is adjustable.

The bending-resistant deep intracranial electrode further comprises a connector 3 connecting the flexible catheter 22 and a shield sleeve 2. The flexible catheter 22 is folded and received in the shield sleeve 2. By pulling out a preset length of the flexible catheter 22 from the shield sleeve 2, a conductor length is varied and a resonant heating of the bending-resistant deep intracranial electrode is reduced.

In the imaging process of magnetic resonance imaging, implanted or semi-implanted medical devices implanted in the body of the patient interacts with the magnetic resonance imaging. The greatest security risk caused by the resonance that the slender conductor structure of the bending-resistant deep intracranial electrode may be heated due to radio frequency induction. Intracranial resonance heating of the implants is very dangerous to the patient's health.

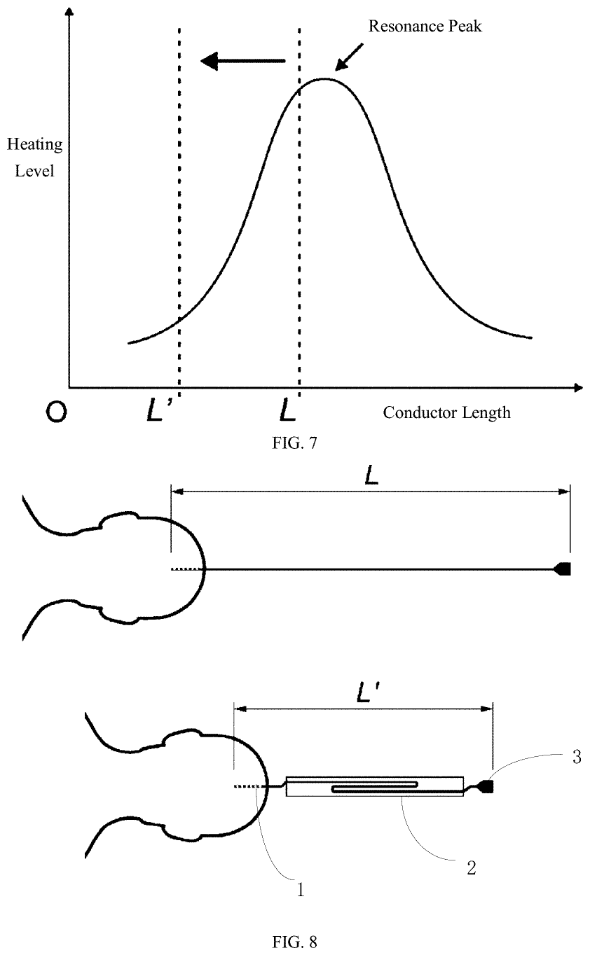

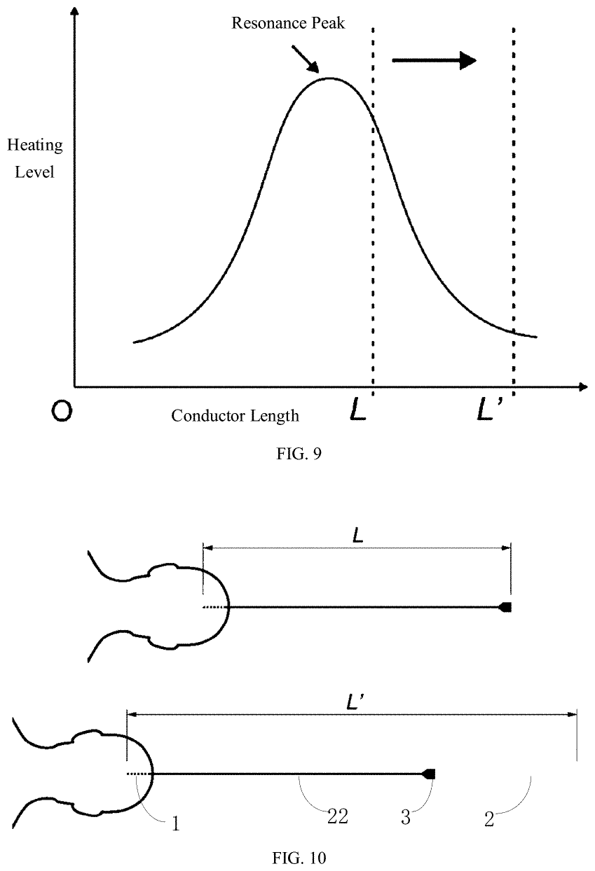

The length of the conductor is analyzed to be an important influence factor for the heating of the conductor of the bending-resistant deep intracranial electrode. Relations between the heating level versus the length of the conductor are as shown in FIGS. 7 and 9. The implanted end of the bending-resistant deep intracranial electrode is heated severely when the length of the conductor is a particular length around a peak value. The particular length is called resonant length. The greater the difference between the overall length of the conductor of the bending-resistant deep intracranial electrode and the resonance length is, the milder the implanted end of the electrode is heated. Therefore, in order to lessen radio-frequency induction heating, the overall length of the electrode conductor should be far different from the resonant length.

However, the length of the electrode conductor is limited by different scenario device performances. A particular value has to be set which is proximate to the resonance length. The resonance length of the electrode conductor is relevant to parameters of the magnetic resonance imaging equipment. For example, for an identical electrode conductor, a resonance length in a 1.5 T magnetic resonance imaging equipment differs from that in a 3.0 T magnetic resonance imaging equipment. In the 1.5 T magnetic resonance imaging equipment, the resonance length of an identical electrode conductor equals to around twice that in the 3.0 T magnetic resonance imaging equipment. Therefore, the length of the electrode conductor in the present embodiment may be designed to be adjustable such as to be compatible with different magnetic resonance imaging equipment.

In the present embodiment, the shield sleeve 2 of the deep intracranial electrode is designed to receive a folded part of the flexible catheter 22, an electrode conductor being installed in the flexible catheter 22. The length of the electrode conductor may be adjusted by changing folded length of the flexible catheter 22 inside the shield sleeve 2. As shown in FIG. 8, a semi-implanted electrode conductor with an original length of L is bent and installed in the shield sleeve 2 capable of shielding magnetic resonance imaging radio-frequency electromagnetic wave.

Referring to FIGS. 7 and 8, the semi-implanted electrode conductor with the original length of L is bent and installed into the shield sleeve 2 which is capable of shielding the magnetic resonance imaging radio-frequency electromagnetic wave. The length of the folded electrode conductor is L′. Since the magnetic resonance imaging radio-frequency electromagnetic wave cannot penetrate the shield sleeve 2, the folded part of the electrode conductor is shielded in the magnetic resonance imaging radio-frequency magnetic field. The conductor with the original length of L is equivalent to the electrode conductor with a length of L′ as shown in FIG. 8, where L′<L. If L′ is further different from the resonance length than L is, the risk of radio-frequency induction heating at the implanted end of electrode conductor can be reduced.

Referring to FIGS. 9 and 10, the length of electrode conductor in the embodiment shown is increased. The semi-implantable electrode conductor with the original length of L is pulled completely out of the shield sleeve 2 which shields the radio-frequency electromagnetic wave, and the length of the electrode conductor after being pulled out completely is L′. As shown in FIG. 10, in this embodiment, the shield sleeve 2 sleeves around an end of the electrode conductor. The shield sleeve 2 may shield a hollow part free of electrode conductor segment at the end, and convert the conductor with the original length of L into an electrode conductor with an equivalent length of L′, in which L′>L. If L′ is further different from the resonance length than L is, the risk of radio-frequency induction heating at the implanted end of electrode conductor can also be reduced.

Therefore, specific lengths of the shield sleeve 2 require to be designed respectively for 1.5 T and 3.0 T magnetic resonance imaging equipment. In the application of 1.5 T and 3.0 T magnetic resonance imaging equipment, equivalent lengths of semi-implanted electrode conductor may be extended or shortened according to the equipment requirements respectively to achieve the purpose of reducing the risk of radio-frequency induction heating.

### The Third Embodiment

This embodiment concerns a detailed description of the bending-resistant deep intracranial electrode in the first embodiment.

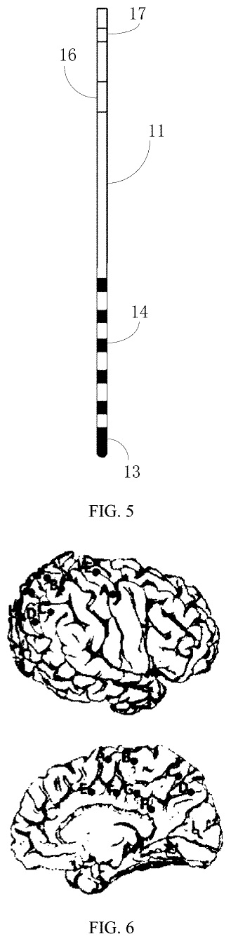

Referring to FIGS. 2 and 5, the majority of the intracranial implanted part of the bending-resistant deep intracranial electrode in this embodiment is an insulated support rod 12, an external part of the support rod is sleeved by a flexible sleeve 11. A plurality of annular electrode contacts 14 are arranged at the end of the support rod 12. The implanted end of the bending-resistant deep intracranial electrode is surgically implanted into the intracranial of the patient, such that the plurality of electrode contacts 14 may directly contact the deep brain tissue of the patient, and detect the deep brain electrophysiological activities of the patient.

The flexible sleeve is provided with a scale 16 and a fixation connection mark 17 at an end external to cranial.

The deep intracranial electrode comprises an intracranial electrode support device 1, a plurality of electrode contacts, a flexible catheter 22, a connector 3 connected to the flexible catheter 22 and a shield sleeve 2. The flexible catheter 22 is an insulated tube.

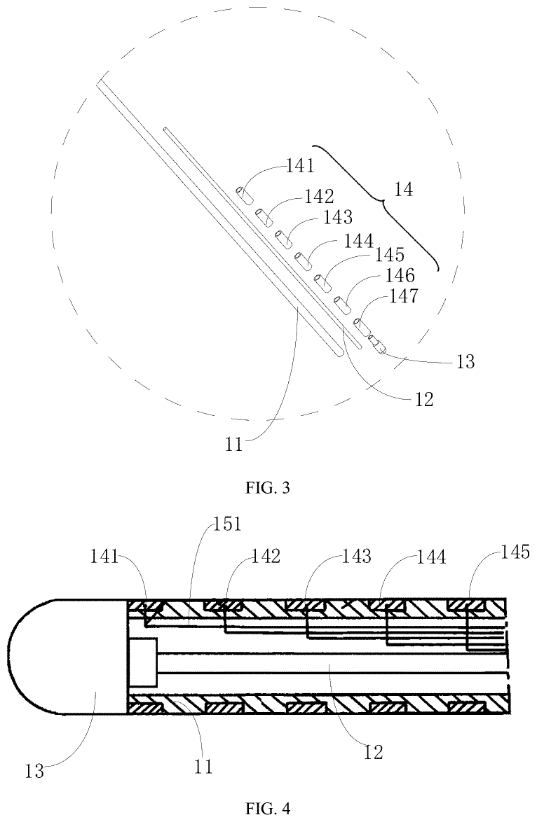

As shown in FIGS. 3 and 4, the plurality of electrode contacts 14 include a first electrode contact 141, a second electrode contact 142, a third electrode contact 143, a fourth electrode contact 144, a fifth electrode contact 145, a sixth electrode contact 146, a seventh electrode contact 147, and an electrode contact head 13.

The intracranial electrode support device 1 comprises an insulated support rod 12 and a flexible sleeve 11.

As shown in FIG. 6, the plurality of electrode contacts is fixed outside the flexible sleeve 11. The support rod 12 is installed inside the flexible sleeve 11, and a gap receiving conducting wires of an electrode conductor, such as the electrode conductor 151, of the plurality of electrode contacts 14 is defined between the support rod 12 and the flexible sleeve 11. The support rod 12 is made of a shape memory alloy material which is subjected to an annealing process and with a preset phase-transformation temperature, such that the support rod 12 may recover to an original shape after being deformed by an external force.

In order to reduce the harm of resonance heating to the patients, the medicinal shape memory alloy material is a non-magnetic shape memory alloy material.

As a first embodiment, the non-magnetic shape memory alloy material is a nickel-titanium shape memory alloy (TiNi). The preset phase-transformation temperature is a first phase-transformation temperature higher than a storage and ambient temperature of the deep intracranial electrodes.

As in another embodiment, the non-magnetic shape memory alloy material is a nickel-titanium shape memory alloy (TiNi). The preset phase-transformation temperature is a second phase-transformation temperature lower than a storage and ambient temperature of the deep intracranial electrodes.

The flexible catheter 22 is folded and received in the shield sleeve 2. By pulling out a preset length of the flexible catheter 22 from the shield sleeve 2, a conductor length is varied and a resonant heating of the bending-resistant deep intracranial electrode is reduced. Detailed implementation is as described in the first embodiment.

A plurality of electrode conducting wires of the plurality of electrode contacts 14 are led out of skull and received in the flexible catheter 22. Along the flexible catheter 22, each electrode contact 14 is electrically connected to corresponding connection terminal of the connector 3 via the electrode conducting wires.

Each connection terminal of the connector 3 is arranged at the electrode conductor inside the flexible catheter, and connected respectively to the electrode contacts 14 of the implanted end. The connector 3 is plugged in the electroencephalograph. The electrophysiological signals collected at the electrode contacts are transmitted to the electroencephalograph through the electrode conductor and the connector 3, and hence forms intracranial electrophysiological images.

In the present embodiment, the intracranial electrode supporting device 1 is fixedly connected to the flexible catheter 22 at the fixation connection mark 17 via the guiding fixing assembly 23.

In localization and fixation of the protrusion head of the implanted end, the guiding fixing assembly 23 comprises a guiding fixing screw and a guiding fixing nut which are for clasping and connecting the support rod 12, the flexible sleeve 11 and the flexible catheter 22.

In order to lessen force applied to the electrode conductor, a tensile fiber is disposed between each electrode contact on the electrode support device and a corresponding connecting terminal of the connector 3. By arranging a slim rope made of a non-stretchable fibrous material inside the tube of the bending-resistant deep intracranial electrode, the slim rope may be fixedly connected to the electrode contacts at two ends of the electrode conductor as well as corresponding connection terminal of the connector 3. When the electrode body is pulled, the slim rope made of the non-stretchable fibrous material may bear the tension, improving tensile strength of the bending-resistant deep intracranial electrode.

At the same time, the length of the flexible catheter 22 is shorter than that of the electrode body inside the flexible catheter.

The flexible catheter 22 adopts a sleeve made of non-stretchable transparent material. One end of the flexible catheter 22 is connected to the connector 3 via a fixed part, the other end fixedly connected to the guiding fixing assembly 23 via the fixation nut. The length of the sleeve is slightly shorter than that of the electrode body inside the tube. While under pulling force, the flexible catheter 22 bears the tension, and the electrode body inside the tube may still keep a loose state all the time and avoid being damaged by the tension.

In the method for manufacturing the deep intracranial electrode, the bending-resistant deep intracranial electrode and the electroencephalograph provided by the embodiments, the support rod 12 in the implanted end of the electrode is made of the shape memory alloy material and provides the bending-resistant deep intracranial electrode with special protective measures, and makes the implanted end of the electrode recover to its original shape after being deformed by external force, improving bending-resistant capability of the implanted end of the electrode and prolonging service life of the medical apparatus and equipment.

In the method for manufacturing the deep intracranial electrode, the bending-resistant deep intracranial electrode and the electroencephalograph provided by the embodiments, the support rod 12 of the implanted end of the electrode is made of a non-magnetic shape memory alloy material, which is compatible with high field magnetic resonance imaging operations, such that the bending-resistant deep intracranial electrode is implanted, at the same time, high field magnetic resonance imaging is performed simultaneously. For example, 3.0 TMRI compatibility is realized, and artifacts in magnetic resonance imaging caused by the electrode is also eliminated.

In the method for manufacturing the deep intracranial electrode, the bending-resistant deep intracranial electrode and the electroencephalograph provided by the embodiments, protection to the electrode conductor is strengthened by various structural design. For example, the length of the flexible catheter 22 is shorter than that of the electrode body inside the tube; furthermore, the tensile fiber is disposed between each electrode contact on the electrode support device and a corresponding connecting terminal of the connector 3. The structures above may avoid electrode from being broken when the patient pull the electrode accidentally in the process of continuous electroencephalography detection.

Above all, it should be noted that the above embodiments are merely for illustrating instead of limiting technical solutions of the present application. The technical features of each of the above embodiments or among different embodiments may also be combined under the principle of the present application. The steps may be implemented in any order, and there exist many alternative variations from different aspects of the present application described above, which are not provided in detail for simplicity purpose. Although the present application is described in detail, those ordinarily skilled in the field shall understand that they may still modify the technical solutions recorded in the foregoing embodiments, or replace some equivalent technical features thereof. Such modifications or replacements do not deviate the principle of corresponding technical solutions from the scope of the technical solutions of each embodiment of the present application.

What is claimed is:

1. A method for manufacturing bending-resistant deep intracranial electrode, comprising:
manufacturing a support rod of a deep intracranial electrode with a shape memory alloy material, the shape memory alloy having a preset phase-transformation temperature;
subjecting the support rod in a straight state to an annealing process such that the support rod memorizes a straight shape.

2. The method for manufacturing bending-resistant deep intracranial electrode of claim 1, wherein the shape memory alloy material is a nickel-titanium shape memory alloy, the preset phase-transformation temperature being a first phase-transformation temperature higher than a storage and ambient temperature of the deep intracranial electrode;
when the deep intracranial electrode is deformed, heating up the deep intracranial electrode to be over the first phase-transformation temperature to recover the support rod of the deep intracranial electrode to the straight shape.

3. The method for manufacturing bending-resistant deep intracranial electrode of claim 1, wherein the shape memory alloy material is a nickel-titanium shape memory alloy, the preset phase-transformation temperature being a second phase-transformation temperature lower than a storage and ambient temperature of the deep intracranial electrode;
when the deep intracranial electrode is deformed, standing the deep intracranial electrode for a preset time to recover the support rod of the deep intracranial electrode to the straight shape.

4. A bending-resistant deep intracranial electrode, comprising an intracranial electrode support device, a plurality of electrode contacts and a flexible catheter, the intracranial electrode support device comprising an insulated support rod and a flexible sleeve, the plurality of electrode contacts fixed outside the flexible sleeve, wherein the support rod is installed inside the flexible sleeve, and a gap receiving conducting wires of the plurality of electrode contacts is defined between the support rod and the flexible sleeve; the support rod is made of a shape memory alloy material which is subjected to an annealing process and with a preset phase-transformation temperature such that the support rod recovers to an original shape after being deformed by an external force.

5. The bending-resistant deep intracranial electrode of claim 4, wherein the shape memory alloy material is a non-magnetic shape memory alloy material.

6. The bending-resistant deep intracranial electrode of claim 5, wherein the shape memory alloy material is a nickel-titanium shape memory alloy, the preset phase-transformation temperature being a first phase-transformation temperature higher than a storage and ambient temperature of the deep intracranial electrode.

7. The bending-resistant deep intracranial electrode of claim 5, wherein the shape memory alloy material is a nickel-titanium shape memory alloy, the preset phase-transformation temperature being a second phase-transformation temperature lower than a storage and ambient temperature of the deep intracranial electrode.

8. The bending-resistant deep intracranial electrode of claim 4, wherein the bending-resistant deep intracranial electrode further comprises a connector connecting the flexible catheter and a shield sleeve, the flexible catheter being folded and received in the shield sleeve; by pulling out a preset length of the flexible catheter from the shield sleeve, a conductor length is varied and a resonant heating of the bending-resistant deep intracranial electrode is reduced.

9. The bending-resistant deep intracranial electrode of claim 8, wherein a plurality of electrode conducting wires of the plurality of electrode contacts are received in the flexible catheter, each electrode contact being electrically connected to corresponding connection terminal of the connector via the electrode conducting wires.

10. The bending-resistant deep intracranial electrode of claim 4, wherein the intracranial electrode supporting device is connected to the flexible catheter via a guiding fixing assembly, the guiding fixing assembly comprising a guiding fixing screw and a guiding fixing nut which are for clasping and connecting the support rod, the flexible sleeve and the flexible catheter.

11. The bending-resistant deep intracranial electrode of claim 8, wherein a tensile fiber is disposed between each electrode contact on the electrode support device and a corresponding connecting terminal of the connector.

12. The bending-resistant deep intracranial electrode of claim 4, wherein a length of the flexible catheter is less than that of an electrode body within the flexible catheter.

13. An electroencephalograph, connected to a plurality of deep intracranial electrodes, each deep intracranial electrode comprising an intracranial electrode support device, a plurality of electrode contacts and a flexible catheter, the intracranial electrode support device comprising an insulated support rod and a flexible sleeve, the plurality of electrode contacts fixed outside the flexible sleeve, wherein the support rod is installed inside the flexible sleeve, and a gap receiving conducting wires of the plurality of electrode contacts is defined between the support rod and the flexible sleeve; the support rod is made of a shape memory alloy material which is subjected to an annealing process and with a preset phase-transformation temperature such that the support rod recovers to an original shape after being deformed by an external force.

14. The electroencephalograph of claim 13, wherein the shape memory alloy material is a non-magnetic shape memory alloy material.

15. The electroencephalograph of claim 14, wherein the shape memory alloy material is a nickel-titanium shape memory alloy, the preset phase-transformation temperature being a first phase-transformation temperature higher than a storage and ambient temperature of the deep intracranial electrodes.

16. The electroencephalograph of claim 14, wherein the shape memory alloy material is a nickel-titanium shape memory alloy, the preset phase-transformation temperature being a second phase-transformation temperature lower than a storage and ambient temperature of the deep intracranial electrodes.

17. The electroencephalograph of claim 13, wherein each of the deep intracranial electrodes further comprises a connector connecting the flexible catheter and a shield sleeve, the flexible catheter being folded and received in the shield sleeve; by pulling out a preset length of the flexible catheter from the shield sleeve, a conductor length is varied and a resonant heating of the bending-resistant deep intracranial electrode is reduced.
